(19) United States
(12) Patent Application Publication
(10) Pub. No.: US 2019/0164634 A1
(43) Pub. Date: May 30, 2019

(54) DATA NOTIFICATION SYSTEM FOR HEALTH DATA

(71) Applicant: International Business Machines Corporation, Armonk, NY (US)

(72) Inventors: Paul R. Bastide, Boxford, MA (US); Senthil Bakthavachalam, Yorktown Heights, NY (US); Rajas H. Karandikar, Indianapolis, IN (US); Yu Gu, Austin, TX (US)

(21) Appl. No.: 15/828,192

(22) Filed: Nov. 30, 2017

(51) Int. Cl. G16H 10/60; G06F 17/30; H04L 29/06

(52) U.S. Cl. CPC G16H 10/60; H04L 63/083; G06F 17/30991; G06F 17/30864

(57) ABSTRACT

Systems, methods, and computer program products to perform an operation comprising receiving a request specifying to receive notifications for updates to data in a multi-tenant data store for a first group comprising a plurality of people, translating the request based on a set of mappings to reflect a physical representation of the data in the data store, generating a notification reflecting an update to data in the data store for a first person of the plurality of people, and storing the notification on a streaming platform for access by a first client application, wherein the notification comprises data describing a location of the updated data in the multi-tenant data store.

### BACKGROUND

The present invention relates to notification systems, and more specifically, to data notifications systems for health data.

Data reservoirs allow all forms of customer-specific data to coexist in a normalized, single storage repository and data processing engine. Data reservoirs are often used as a data source for multi-dimensional analytics programs. To access the data stored in data reservoirs, applications must continuously fetch the data from the data reservoir in batch operations, and update their own data stores accordingly. However, the continual batch loading of large data sets from the data reservoir is inefficient. Similarly, if the application has to poll the data reservoir for new updates, the poll interval affects the turnaround time to implement advanced solutions.

### SUMMARY

According to one embodiment, a method comprises receiving a request specifying to receive notifications for updates to data in a multi-tenant data store for a first group comprising a plurality of people, translating the request based on a set of mappings to reflect a physical representation of the data in the data store, generating a notification reflecting an update to data in the data store for a first person of the plurality of people, and storing the notification on a streaming platform for access by a first client application, wherein the notification comprises data describing a location of the updated data in the multi-tenant data store.

In another embodiment, a system comprises a processor and a memory storing instructions, which when executed by the processor, performs an operation comprising receiving a request specifying to receive notifications for updates to data in a multi-tenant data store for a first group comprising a plurality of people, translating the request based on a set of mappings to reflect a physical representation of the data in the data store, generating a notification reflecting an update to data in the data store for a first person of the plurality of people, and storing the notification on a streaming platform for access by a first client application, wherein the notification comprises data describing a location of the updated data in the multi-tenant data store.

In another embodiment, a computer-readable storage medium has computer-readable program code embodied therewith, the computer-readable program code executable by a processor to perform an operation comprising receiving a request specifying to receive notifications for updates to data in a multi-tenant data store for a first group comprising a plurality of people, translating the request based on a set of mappings to reflect a physical representation of the data in the data store, generating a notification reflecting an update to data in the data store for a first person of the plurality of people, and storing the notification on a streaming platform for access by a first client application, wherein the notification comprises data describing a location of the updated data in the multi-tenant data store.

### BRIEF DESCRIPTION OF THE SEVERAL VIEWS OF THE DRAWINGS

FIGS. 1A-1B illustrate a data notification system for health data, according to various embodiments.

FIGS. 2A-2C illustrate examples of notification data provided by a data notification system, according to various embodiments.

FIG. 3 is a flow chart illustrating a method to provide a data notification system for health data, according to one embodiment.

FIG. 4 is a flow chart illustrating a method to create a topic and secure data, according to one embodiment.

FIG. 5 is a flow chart illustrating a method to provide push notifications for updates of data, according to one embodiment.

FIG. 6 illustrates a data notification system for health data, according to one embodiment.

### DETAILED DESCRIPTION

Embodiments disclosed herein provide data notification systems for real-time health data. Generally, a user or application may specify an abstract request to monitor the data of a group of people, e.g., “all people between the ages of 20-39”. The data notification system may translate the abstract request to conform with a corresponding backend database that stores the health data for the people. Doing so allows the data notification system to be agnostic to any particular backend database, allows the backend database to be changed as needed, and does not require the backend database schema to be disclosed to clients.

Responsive to the request, the data notification system may further provision a “topic” for the requested group of people in a notification platform. When updates are made to the health data of one member of the group, the data notification system determines which topics should receive an indication of the update. The data notification system provides an indication of the update to each topic in the notification platform, which retains the information in order for each topic. A client application may then authenticate using a secure key, and receive the indication of the data updates from the notification platform. The client application may use the received information to generate a query to a location where the updated data is stored. The client application may then issue the query, and receive the updated data from the storage location.

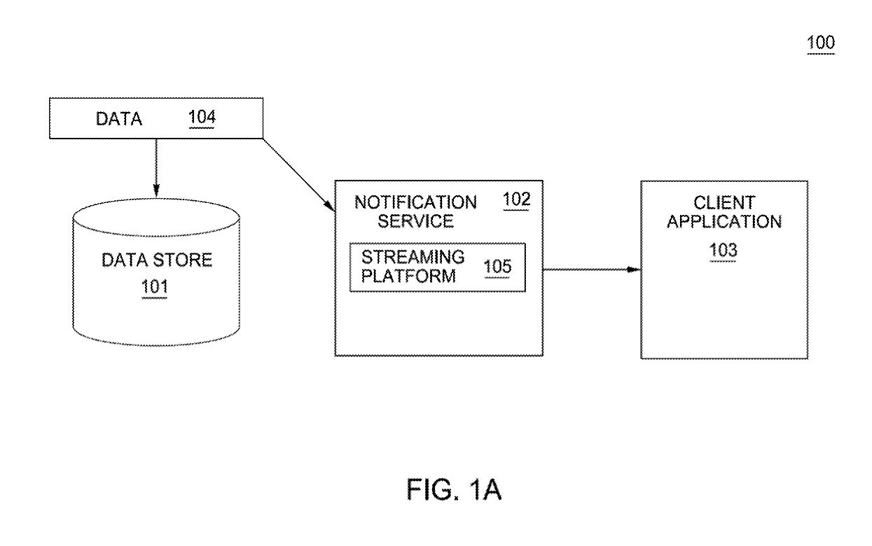

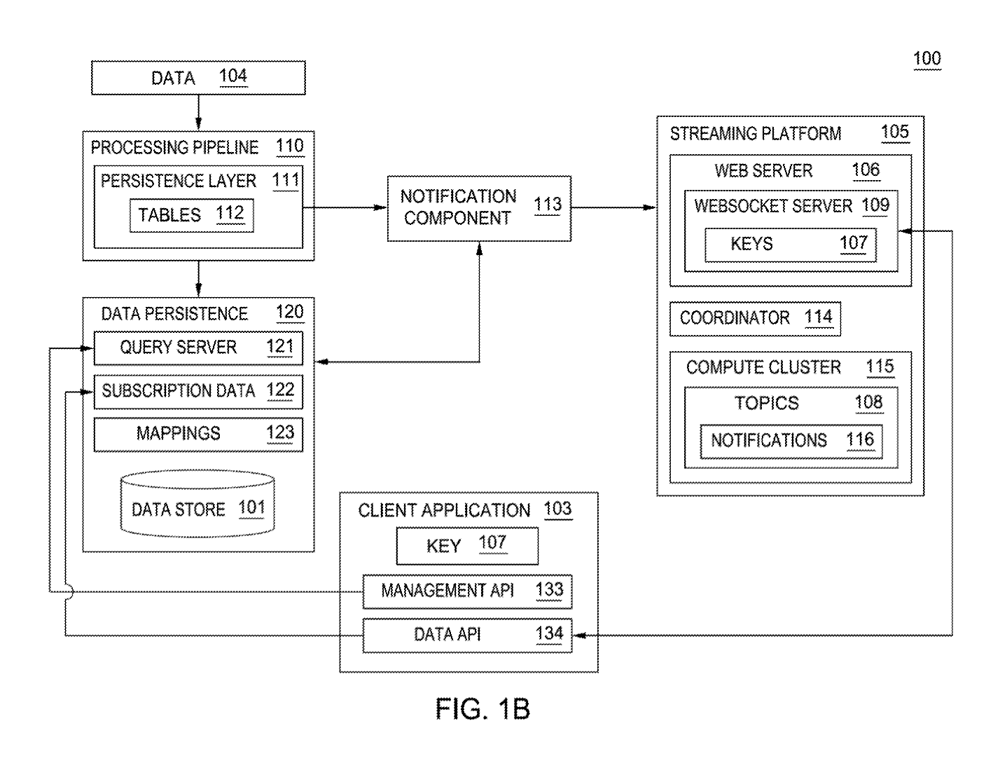

FIG. 1A illustrates components of a data notification system 100 for health data, according to one embodiment. As shown, the system 100 includes a data store 101, a notification service 102, and a client application 103. The data store 101 is representative of any number and type of multi-tenant data stores, such as a relational database, a data reservoir, a cloud storage cluster, and the like. Generally, the data store 101 stores data related to a plurality of people, including medical and/or health data. For example, the data store 101 may include data for different hospitals and include patient medical records, patient profiles, doctor notes, test results, and the like, for each hospital. As such, the data in the data store 101 may be updated at any given time. Data 104 reflects an example update of the data in the data store 101. The update may correspond to any type of data update, such as storing an indication of a patient taking a blood test, storing an indication of the results of the blood test, and/or a doctor's note generated based on the results of the blood test.

When the data update 104 occurs, the data store 101 may be modified any number of times to reflect the data update 104. However, the client application 103 must be made aware of the data update 104. Advantageously, an indication of the data update 104 is provided to the notification service 102. The notification service 102 includes a streaming platform 105, which allows publishers to publish streams of records (e.g., data updates), and allows subscribers (e.g., the client application 103) to consume the streams of records. One example of a streaming platform is Kafka® by Apache®. The subscriber (e.g., a client application 103) may specify an abstract request to receive notifications for updates to the data store 101. The abstract request may specify a group of people to monitor, e.g., “female non-smokers between the ages of 40-50”. When data in the data store 101 is updated for the specified group of people, an indication of each update is stored in order (based on a respective timestamp) in a “topic” in the streaming platform 105 (e.g., a topic allocated to the requested female non-smokers between the ages of 40-50). The notification service 102 and/or the streaming platform 105 may then transmit an indication to the client application 103 that an update is available. The client application 103 may authenticate with the streaming platform 105 using a secure key. The streaming platform 105 may then transmit, for each notification, information that allows the client application 103 to build a query to access the updated data in the data store 101. For example, the information may include a tenant identifier, a table identifier, and a patient identifier. The client application 103 may then generate a query targeting the data store 101, which, when processed, returns the requested data to the client application 103.

FIG. 1B illustrates the data notification system 100 in greater detail, according to one embodiment. As shown, an example client application 103 includes a key 107, a management application programming interface (API) 133, and a data API 134. The key 107 is a cryptographic key used to authenticate the client application 103 and secure data transmissions in the system 100. The management API 132 allows the client application 103 to subscribe to updates, cancel subscriptions to updates, and otherwise manage updates received from the streaming platform 105 via the data persistence server 120. When the client application 103 issues a request to the data persistence server 120 to subscribe to updates for a group of people, the client application 103 provides the key 107. The key 107 is stored in the subscription data 122 along with an indication of the topic 116 generated for the request in the streaming platform 105. In at least one embodiment, the data persistence server 120 translates the group specified in the request based on mappings 123 that map abstract entities specified in a request to physical entities in the data store 101. For example, a mapping 123 may map different terms describing a person table (e.g., person_table, persontable, etc.) to the actual person table (e.g., “person_table”) for a given tenant in the data store 101.

The data persistence server 120 may also generate one or more topics 108 in the streaming platform 105 based on the request received from the management API 133 of the client application 103. An indication of each topic 108 is also stored in the subscription data 122. The indication of the topic 108 in the subscription data 122 may be associated with the key 107, an indication of any targeted tables in the data store 101, any conditions (e.g., people between the ages of 10 and 20), and any filtering rules (e.g., do not trigger a notification 116 until a “patient discharge” is stored in a specific location of the data store 101).

As shown, the streaming platform 106 includes one or more web servers 106, which communicate with other applications (e.g., the client application 103) using the hypertext transfer protocol (HTTP). The web servers 106 include one or more websocket servers 109, which provides a full-duplex communications channel between the streaming platform 105 and the client application 103. The websocket servers 109 allow the client application 103 to send messages to the streaming platform 105 and receive notifications from the streaming platform 105 without the streaming platform 105 having to poll the streaming platform 105 for a reply.

The web server 106 and/or the websocket server 109 may authenticate the client application 103 based on the corresponding key 107 (e.g., using transport layer security). Once authenticated, the websocket server 109 may determine, from the coordinator 114, which topics 108 the client application 103 is subscribed. The coordinator 114 generally manages the topics 108 and stores indications of which client applications 103 are subscribers to a given topic 108, which are stored by a plurality of compute nodes of a compute cluster 115. Furthermore, a given subscription may apply to different topics 108 (e.g., a topic 108 for males over 70 with heart disease, and a topic 108 for females over 75 with heart disease). Once the websocket server 109 authenticates the client application 103 and determines which topics 108 the client application 103 is subscribed to, the websocket server 109 pushes an indication of each corresponding notification 116 to the client application 103. As previously stated, the notification 116 includes information that allows the client application to generate a query to retrieve the data from the data store 101. For example, the notification 116 may specify the tenant ID, table ID, and patient (or person) ID. The client application 103 may then generate the query, which is transmitted to the query server 121. The query server 121 processes the query against the data store 101, and returns the data to the client application 103 via the data API 133. The client application 103 may then perform further processing on the received data, e.g., by updating healthcare data models based on the received data.

FIG. 1B further depicts an example processing pipeline 110 that process the data 104 which results in an update, addition, and/or removal to data in the data store 101. The data 104 may be any type of data, such as Health-Level 7 (HL7) data. For example, the processing pipeline 110 may modify data, generate new data, delete data from the data store 101, and the like. As shown, the processing pipeline 110 includes a persistence layer 111, which includes a plurality of tables 112 that store data being processed by the processing pipeline 110. For example, if the data 104 corresponds to a patient's pulse value (e.g., 70 beats per minute), the pulse value may be stored in different tables 112 in the persistence layer 111, such as a general table 112 that stores patient pulse readings, a table 112 for patients with heart disease, and the like. The tables 112 in the persistence layer 111 may be tagged with metadata defining one or more associated topics 108. Once processed, the processing pipeline 110 places the processed data in the data store 101.

The notification component 113 is configured to generate notifications 116 based on updates to data generated by the processing pipeline 110. The notification component 113 may monitor the processing pipeline 110 for data that is modified, and/or the persistence layer 111 may provide an indication of data modification to the notification component 113. In response to data being updated, the notification component 113 receives the subscription data 122 from the data persistence server 120 to identify any topics 108 in the streaming platform 105 that should receive a notification 116 corresponding to the data update. Continuing with the previous example, the subscription data 122 may specify that a first client application 103 is subscribed to the general pulse topic 108, and a second client application 103 is subscribed to the topic 108 for pulse data of patients with heart disease. The notification component 113 may then generate the notification 116 for each topic 108, encrypt the notifications 116 based on the key 107 for each topic 108, and transmit the encrypted notifications 116 to the streaming platform 105, where the notifications 116 are stored in the topics 108. As previously stated, the notifications 116 are stored in the topics 108 in order based on the time of each data update, which may be specified as a timestamp in each notification 116. The streaming platform 105 then allows the client applications 103 to consume the notifications 116 as described above. If, for example, the client application 103 consumes 10 of 20 notifications 116, when the client application 103 sends a request to consume additional notifications 116, the websocket server 109 begins transmission of the 11^{th} notification, and holds all notifications 116 until each notification 116 is consumed by the client application 103.

In at least one embodiment, the notification component 113 computes a score for one or more data updates before generating a notification 116. If the computed score exceeds a corresponding threshold, the notification component 113 generates the notification 116. For example, if a data update specifies that a patient has scheduled a same-day appointment with a physician, the notification component 113 may compute a score that does not exceed a threshold. However, once the visit is completed, the notification component 113 may compute a score that exceeds the threshold, and the notification 116 is generated. The score may be computed based on a function that considers predefined weights for each of a plurality of different events that may cause data updates (e.g., a first weight for scheduling an appointment, a second weight for attending the appointment, a third weight for a test performed during the appointment, etc.).

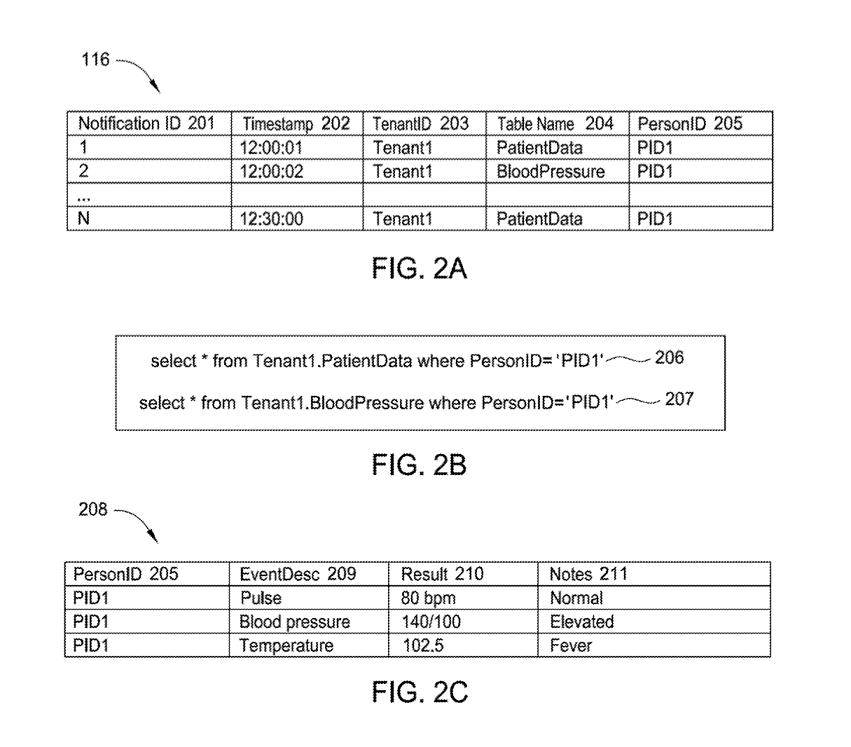

FIG. 2A illustrates a table of example notifications 116 provided by the data notification system 100, according to one embodiment. In the example depicted in FIG. 2A, the notifications 116 belong to a single topic 108 stored by the compute cluster 115 of the streaming platform 105. As shown, each notification in the notifications 116 includes a notification ID 201, a timestamp 202, a tenant ID 203, a table name 204, and a person ID 205. The notification ID 201 is a unique identifier for each notification 116 in the topic 108. The timestamp 202 corresponds to a time when the patient event, data update, and/or notification 116 occurred. The tenant ID 203 corresponds to a tenant identifier in the multi-tenant data store 101. The table name 204 corresponds to a table in the data store 101 where the updated data is stored. The person ID 205 is an identifier corresponding to an individual that is subject to the data update.

The client application 103 may invoke the data API 134 to receive the notifications 116 via the streaming platform 105. The data API 134 and the streaming platform 105 may implement TLS authentication using the key 107 of the client application 107. Once authenticated, the websocket server 109 transmits the notifications 116 to the client application 107. As previously stated, the notification component 113 generates notifications 116 that include enough information to allow a client application 103 to generate a query to access the data from the data store 101. FIG. 2B depicts an example two example queries 206, 207 generated by the client application 103 based on the notifications 116. For example, the client application 103 may generate the query 206 on the notification 116 having the notification ID 201 value of “1” received from the streaming platform 105. As shown, the example query 206 specifies to retrieve data for an example person having a personID of ‘PID1” from the “PatientData” table of the tenant having a tenantID of “Tenant1”. In addition, based on the notification 116 received from the streaming platform 105 having the notification ID of “2”, the client application 103 may generate the query 207 specifying “select * from Tenant1.BloodPressure where PersonID=‘PID1’” to retrieve data from the BloodPressure table of the data store 101. The data API 134 may submit the queries 206, 207 to the query server 121 of the data persistence server 120. The query server 121 may execute the queries 206, 207 against the data store 101 and return a result set to the client application 103.

FIG. 2C depicts an example result set 208 returned by the query server 121 based on processing the queries 206, 207 against the data store 101. As shown, the result set 208 includes a PersonID column 205, an event description column 209, a result column 210, and a notes column 211. The event description column 209 includes an example description of a medical procedure and/or event incurred by the patient having the patient ID 205 of “PID1”. The result column 210 specifies a result, or value, associated with the corresponding event, while the notes column 211 includes a note based on a medical professional's analysis of the result 210. Therefore, as shown, the patient had a pulse reading of 80 beats per minute, which the professional stated was a normal pulse value. Similarly, the patient had a blood pressure reading of 140/100 which was determined to be elevated. The data presented in FIGS. 2A-2C is for illustrative purposes and should not be considered limiting of the disclosure, as any type of data may be stored in a notification 116, query 206-207, and result set 208.

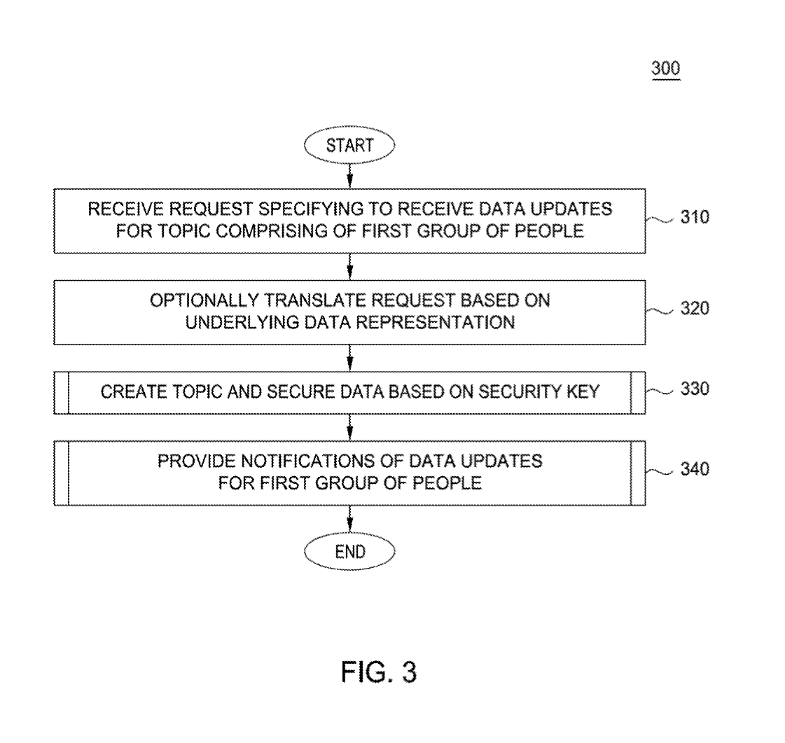

FIG. 3 is a flow chart illustrating a method 300 to provide a data notification system for health data, according to one embodiment. As shown, the method 300 begins at block 310, where the persistence server 120 receives a request from a client application 103 to register for updates to data in the data store 101 for a group of people. The request may specify an abstract definition of the group of people, such as “non-smokers over the age of 60.” The request may further specify a security key 107 of the application. At block 320, the persistence server 120 may optionally translate the request to a physical representation of the data in the data store 101 based on one or more mappings 123. For example, the persistence server 120 may determine a tenant identifier in the data store 101, determine one or more associated tables in the data store 101, representations of age values in the determined tables, and representations of non-smoker status in the determined tables. The data persistence server 120 may then store an indication of the subscription in the subscription data 122.

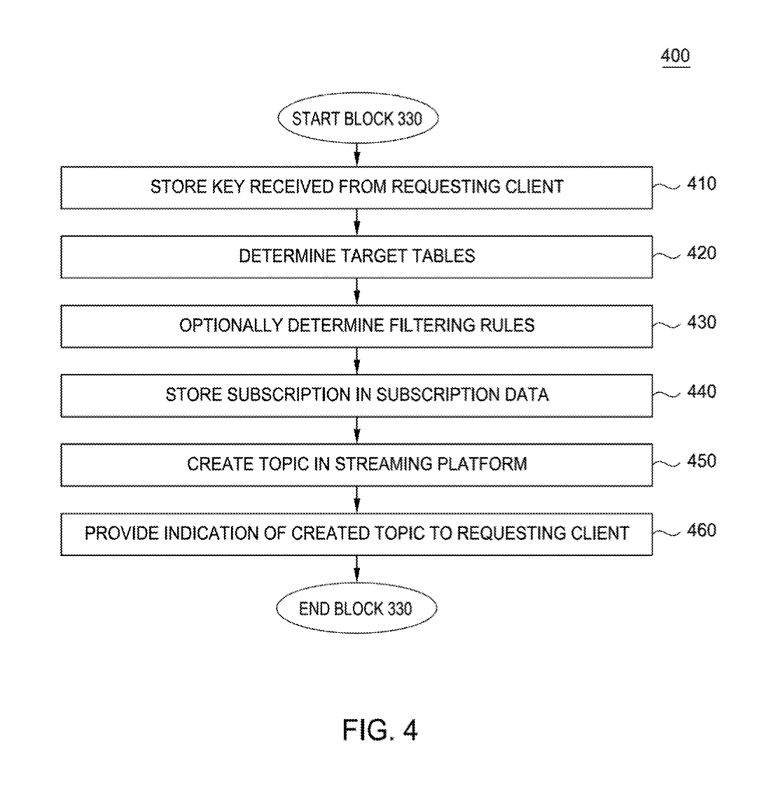

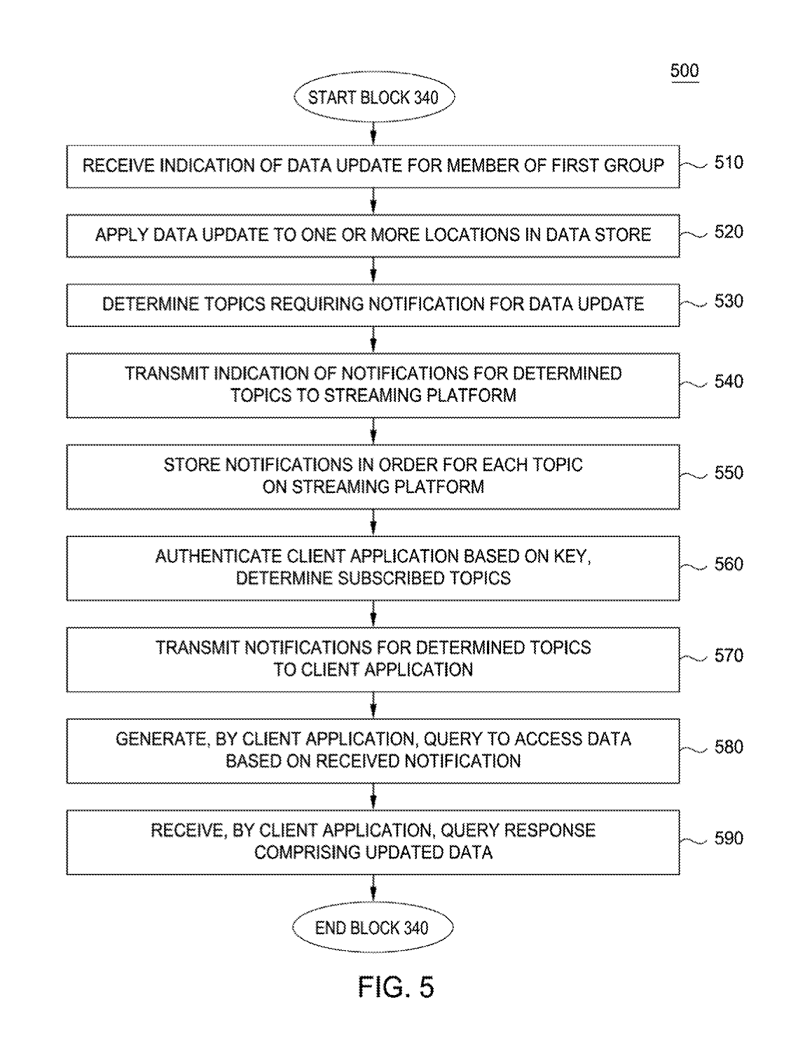

At block 330, described in greater detail with reference to FIG. 4, one or more topics 108 for the subscription are created in the streaming platform 105. For example, the data persistence server 120 may provide an indication of the subscription to the notification component 113 and/or the streaming platform 115, which may generate one or more topics 108 for the subscription. The data persistence server 120, the notification component 113, and the streaming platform 105 may further apply the received key 107 to secure the health data of the associated patients. At block 340, described in greater detail with reference to FIG. 5, the streaming platform 105 provides notifications to the client application 105 describing data updates for the first group of people. For example, if one of the monitored non-smokers is prescribed a new medication, the notification component 113 may generate a notification 116 which serves as a “pointer” to the data in the data store 101 reflecting the prescription. The client application 103 may receive the notification 116 from the streaming platform 105, and generate a query to access the data in the data store 101. The client application 103 may then store the received data and/or perform one or more operations on the received data.

FIG. 4 is a flow chart illustrating a method 400 corresponding to block 330 to create a topic and secure data, according to one embodiment. As shown, the method 400 begins at block 410, where one or more of the data persistence server 120, the notification component 113, and/or the streaming platform 105 stores the security key 107 provided by the client application 103 with the request. At block 420, the data persistence server 120 determines the relevant tables in the data store 101 targeted by the request, e.g., based on a mapping between terms in the request and the physical data representation of the data store 101. In at least one embodiment, the data persistence server 120 applies one or more natural language processing algorithms to a text-based request to extract concepts from the request that can be mapped to physical entities in the data store 101 using the mappings 123.

At block 430, the data persistence server 120 determines whether the request specifies one or more filtering rules. The filtering rules may specify conditions for generating notifications 116. For example, a filtering rule may specify to generate a notification 116 only when a specific type of message is received for a particular patient. As such, the notification component 113 may generate the notification 116 only when this type of message is received in the processing pipeline 110. Similarly, the filtering rules may specify notification timing (e.g., hold all notifications for delivery on an hourly, daily, or weekly basis). Further still, the filtering rules may specify thresholds for any scores generated by the notification component 113 in determining whether to generate a notification 116. At block 440, the data persistence server 120 stores each parameter associated with the subscription in the subscription data 122. At block 450, the streaming platform 105 generates one or more topics 108 for the subscription in the streaming platform 105. At block 460, the streaming platform 105 provides an indication (e.g., an identifier) of the topics 108 created at block 450 to the client application 103. Doing so allows the client application 103 to subsequently request notifications 116 for one, several, or all topics 108 that the client application 108 is subscribed to.

FIG. 5 is a flow chart illustrating a method 500 corresponding to block 340 to provide push notifications for updates of data, according to one embodiment. As shown, the method 500 begins at block 510, where the notification component 113 receives an indication of an update to the data of a member of the first group of people specified in the request received at block 310. For example, results of a blood test of a patient in the first group of people may have been received in the processing pipeline 110. At block 520, the updates are applied to one or more tables (e.g., in the tables 112 of the persistence layer and/or the data store 101). As previously stated, data related to a single event (e.g., a blood pressure reading) may be stored in any number of locations. As such, the notification component 113 may need to generate a notification 116 for one or more of the locations where the data is stored. At block 530, the notification component 113 leverages the subscription data 122 to determine the subscriptions and/or topics 108 that require a notification for the updated data. For example, the subscription data 122 may specify a first, second, and third topic 108 are associated with a client application 103 that has subscribed to data updates for the updated data. Before generating a notification 116, notification component 113 may also determine whether any filtering rules are satisfied by the data updates (e.g., whether the data update is of a specified type). Similarly, the notification component 113 may compute a score for the data updates, and determine whether the score exceeds a corresponding threshold before generating the notification 116.

At block 540, the notification component 113 generates and transmits a notification 116 for each topic 108 identified at block 530. At block 550, the notifications 116 are received by the streaming platform 105 and stored in the associated topics 108. At block 560, the streaming platform 105 receives a request from the client application 103 to receive notifications 116 for the subscribed topics 108. The streaming platform 105 may authenticate the client application 103 using TLS authentication based on the security key 107 of the client application 103. At block 570, the websocket server 109 transmits notifications 108 for each subscribed topic 108 to the client application 103. As previously stated, the notifications 116 include enough information that allows the client application 103 to generate a query to access the data in the data store 101. At block 580, the client application 103 generates one or more queries specifying to access the data in the data store 101 based on the received notifications 116. The data persistence server 120 may authenticate the client application 103 using TLS authentication based on the security key 107 of the client application 103. At block 590, the query server 121 processes the queries and returns the data from the data store 101 to the client application 103.

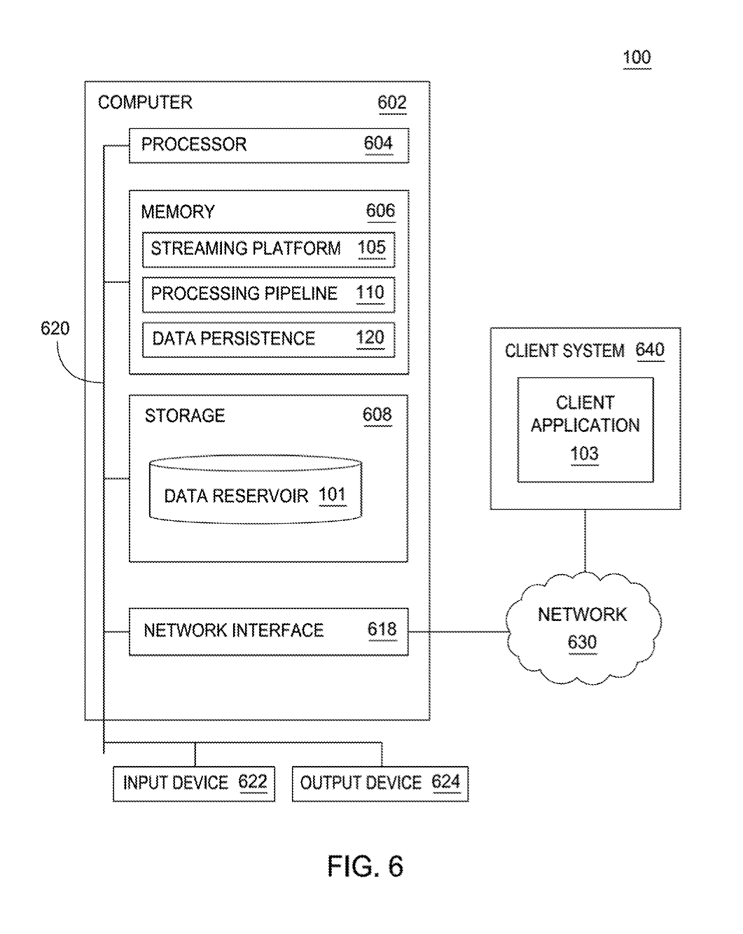

FIG. 6 illustrates the data notification system 100 for health data, according to one embodiment. The system 100 includes a computer 602. The computer 602 may also be connected to other computers, such as the client system 640, via a network 630. In general, the network 630 may be a telecommunications network and/or a wide area network (WAN). In a particular embodiment, the network 630 is the Internet. Although depicted as a single computer 602, the system 100 may generally include any number of computers 602.

The computer 602 generally includes a processor 604 which obtains instructions and data via a bus 620 from a memory 606 and/or a storage 608. The computer 602 may also include one or more network interface devices 618, input devices 622, and output devices 624 connected to the bus 620. The computer 602 is generally under the control of an operating system (not shown). Examples of operating systems include the UNIX operating system, versions of the Microsoft Windows operating system, and distributions of the Linux operating system. (UNIX is a registered trademark of The Open Group in the United States and other countries. Microsoft and Windows are trademarks of Microsoft Corporation in the United States, other countries, or both. Linux is a registered trademark of Linus Torvalds in the United States, other countries, or both.) More generally, any operating system supporting the functions disclosed herein may be used. The processor 604 is a programmable logic device that performs instruction, logic, and mathematical processing, and may be representative of one or more CPUs. The network interface device 618 may be any type of network communications device allowing the computer 602 to communicate with other computers via the network 630.

The storage 608 is representative of hard-disk drives, solid state drives, flash memory devices, optical media and the like. Generally, the storage 608 stores application programs and data for use by the computer 602. In addition, the memory 606 and the storage 608 may be considered to include memory physically located elsewhere; for example, on another computer coupled to the computer 602 via the bus 620.

The input device 622 may be any device for providing input to the computing system 602. For example, a keyboard and/or a mouse may be used. The input device 622 represents a wide variety of input devices, including keyboards, mice, controllers, and so on. Furthermore, the input device 622 may include a set of buttons, switches or other physical device mechanisms for controlling the computer 602. The output device 624 may include output devices such as monitors, touch screen displays, and so on.

As shown, the memory 606 contains the, streaming platform 105, processing pipeline 110, and the data persistence server 120, each described in greater detail above. As shown, the storage 608 contains the data reservoir 101, described in greater detail above. Generally, the system 100, including the computer 602, is configured to implement all systems, methods, and functionality described above with reference to FIGS. 1-5. The client system 640 executes the client application 103 and similarly includes a processor, memory, storage, network interface, bus, input device, and output device, each not pictured for clarity.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the foregoing, reference is made to embodiments presented in this disclosure. However, the scope of the present disclosure is not limited to specific described embodiments. Instead, any combination of the recited features and elements, whether related to different embodiments or not, is contemplated to implement and practice contemplated embodiments. Furthermore, although embodiments disclosed herein may achieve advantages over other possible solutions or over the prior art, whether or not a particular advantage is achieved by a given embodiment is not limiting of the scope of the present disclosure. Thus, the recited aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

Aspects of the present invention may take the form of an entirely hardware embodiment, an entirely software embodiment (including firmware, resident software, microcode, etc.) or an embodiment combining software and hardware aspects that may all generally be referred to herein as a “circuit,” “module” or “system.”

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g. an amount of storage space consumed by a user or a number of virtualized systems instantiated by the user). A user can access any of the resources that reside in the cloud at any time, and from anywhere across the Internet. In context of the present invention, a user may access applications or related data available in the cloud. For example, the notification component 113 could execute on a computing system in the cloud and generate notifications 116. In such a case, the streaming platform 105 may store the generated notifications 116 at a storage location in the cloud. Doing so allows a user to access this information from any computing system attached to a network connected to the cloud (e.g., the Internet).

While the foregoing is directed to embodiments of the present invention, other and further embodiments of the invention may be devised without departing from the basic scope thereof, and the scope thereof is determined by the claims that follow. cm What is claimed is:

1. A method, comprising:
receiving a request specifying to receive notifications for updates to data in a multi-tenant data store for a first group comprising a plurality of people;
translating the request based on a set of mappings to reflect a physical representation of the data in the data store;
generating a notification reflecting an update to data in the data store for a first person of the plurality of people; and
storing the notification on a streaming platform for access by a first client application, wherein the notification comprises data describing a location of the updated data in the multi-tenant data store.

2. The method of claim 1, wherein the request is received from a client application, wherein the data describing the location of the updated data comprises: (i) a tenant identifier associated with the client application in the multi-tenant data store, (ii) a database table storing the updated data in the multi-tenant data store, and (iii) a key value associated with the first person in the database table.

3. The method of claim 2, further comprising:
receiving, by a query server from the client application, a query targeting the data store based on the data describing the location of the updated data;
processing the received query against the data store; and
returning a result set to the client application as responsive to the query.

4. The method of claim 3, wherein the notification is associated with a first topic, of the plurality of topics managed by the streaming platform, wherein the streaming platform stores the notifications for consumption by the client application, wherein the notification and the updated data are secured based on a security key provided in the request.

5. The method of claim 4, further comprising:
determining that the update to the data has been generated in a processing pipeline; and
determining that the first topic is associated with updates to the updated data.

6. The method of claim 5, further comprising:
receiving, by the streaming platform, an indication from the client application to access the notification;
authenticating the client application based on the security key; and
transmitting, by a websocket server, the notification to the client application.

7. The method of claim 4, further comprising:
determining that a second topic is associated with updates to the updated data;
generating a second notification reflecting the update to the updated data for the second topic; and
storing, by the streaming platform, the second notification in the second topic.

8. A computer program product, comprising:
a non-transitory computer-readable storage medium having computer readable program code embodied therewith, the computer readable program code executable by a processor to perform an operation comprising:
receiving a request specifying to receive notifications for updates to data in a multi-tenant data store for a first group comprising a plurality of people;
translating the request based on a set of mappings to reflect a physical representation of the data in the data store;
generating a notification reflecting an update to data in the data store for a first person of the plurality of people; and
storing the notification on a streaming platform for access by a first client application, wherein the notification comprises data describing a location of the updated data in the multi-tenant data store.

9. The computer program product of claim 8, wherein the request is received from a client application, wherein the data describing the location of the updated data comprises: (i) a tenant identifier associated with the client application in the multi-tenant data store, (ii) a database table storing the updated data in the multi-tenant data store, and (iii) a key value associated with the first person in the database table.

10. The computer program product of claim 9, the operation further comprising:
receiving, by a query server from the client application, a query targeting the data store based on the data describing the location of the updated data;
processing the received query against the data store; and
returning a result set to the client application as responsive to the query.

11. The computer program product of claim 10, wherein the notification is associated with a first topic, of the plurality of topics managed by the streaming platform, wherein the streaming platform stores the notifications for consumption by the client application, wherein the notification and the updated data are secured based on a security key provided in the request.

12. The computer program product of claim 11, the operation further comprising:
determining that the update to the data has been generated in a processing pipeline; and
determining that the first topic is associated with updates to the updated data.

13. The computer program product of claim 13, the operation further comprising:
receiving, by the streaming platform, an indication from the client application to access the notification;
authenticating the client application based on the security key; and
transmitting, by a websocket server, the notification to the client application.

14. The computer program product of claim 11, further comprising:
determining that a second topic is associated with updates to the updated data;
generating a second notification reflecting the update to the updated data for the second topic; and
storing, by the streaming platform, the second notification in the second topic.

15. A system, comprising:
a processor; and
a memory storing one or more instructions which, when executed by the processor, performs an operation comprising:
receiving a request specifying to receive notifications for updates to data in a multi-tenant data store for a first group comprising a plurality of people;
translating the request based on a set of mappings to reflect a physical representation of the data in the data store;
generating a notification reflecting an update to data in the data store for a first person of the plurality of people; and
storing the notification on a streaming platform for access by a first client application, wherein the notification comprises data describing a location of the updated data in the multi-tenant data store.

16. The system of claim 15, wherein the request is received from a client application, wherein the data describing the location of the updated data comprises: (i) a tenant identifier associated with the client application in the multi-tenant data store, (ii) a database table storing the updated data in the multi-tenant data store, and (iii) a key value associated with the first person in the database table.

17. The system of claim 16, the operation further comprising:
receiving, by a query server from the client application, a query targeting the data store based on the data describing the location of the updated data;
processing the received query against the data store; and
returning a result set to the client application as responsive to the query.

18. The system of claim 17, wherein the notification is associated with a first topic, of the plurality of topics managed by the streaming platform, wherein the streaming platform stores the notifications for consumption by the client application, wherein the notification and the updated data are secured based on a security key provided in the request.

19. The system of claim 18, the operation further comprising:
determining that the update to the data has been generated in a processing pipeline; and
determining that the first topic is associated with updates to the updated data.

20. The system of claim 15, the operation further comprising:
receiving, by the streaming platform, an indication from the client application to access the notification;
authenticating the client application based on the security key; and
transmitting, by a websocket server, the notification to the client application.
